(19) United States
(12) Patent Application Publication
(10) Pub. No.: US 2007/0160974 A1
(43) Pub. Date: Jul. 12, 2007

(54) Human embryonic stem cell clones

(75) Inventors: Kuldip S. Sidhu, West Pennant Hills (AU); Bernard E. Tuch, Maroubra (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: South Eastern Sydney and Illawarra Area Health Service, Wollongong (AU)

(21) Appl. No.: 11/474,059

(22) Filed: Jun. 22, 2006

(30) Foreign Application Priority Data
Jan. 10, 2006 (AU) 2006900111

(51) Int. Cl. C12Q 1/00; C12N 5/08

(52) U.S. Cl. 435/4; 435/366

(57) ABSTRACT

The present invention provides methods for isolating individual viable stem cells from a stem cell line, methods for deriving one or more clones from a stem cell line, individual viable cells and clones derived from stem cell lines by the methods disclosed, methods for producing differentiated cells from the individual viable cells and clones so derived, differentiated cells so produced, methods for treating diseases using the cells and clones described herein and methods for proliferating cells and clones in undifferentiated form.

### TECHNICAL FIELD

The present invention relates generally to methods for the isolation of viable individual cells from a stem cell line, such as an embryonic stem cell line. The present invention further relates to three novel human embryonic stem cell clones isolated by the novel methodology and to uses thereof.

### BACKGROUND OF THE INVENTION

Stem cells are distinguishable from other cell types in that they are capable of both differentiating into specialized cells and dividing continuously for long periods of time, thus making them suitable as cell lines in research. They are found in embryonic, fetal and adult tissues.

Cells comprising a human embryo up to the 8 cell stage are “totipotent”, each being capable of developing into an entire human being. As the cells of an embryo continue to divide, they form a blastocyst, being a hollow sphere of about 120 cells with an outer layer and an inner cell mass. The outer layer develops into the placenta while the inner cell mass comprises embryonic stem cells ESCs which are “pluripotent”, being capable of differentiating into all cell types found in a human body. However, as pluripotent ESCs cannot develop into tissues necessary to support pregnancy, such as the placenta, ESCs cannot of themselves develop into a human being.

Following the successful derivation of five human embryonic stem cell (hESC) lines in 1998, many new hESC lines have been created around the world. To date, it is estimated that at least 225 new hESC lines have been produced world-wide, of which 78 are currently listed in the National Institute Health (NIH) registry. Of these 78 lines, only about 26 have been characterized to varying degrees and are available for research. In addition, many of these hESC lines are not clonal, were derived under different culture conditions and were propagated on different feeder layers such as mouse embryonic fibroblasts (MEF), STO (an immortal mouse embryonic fibroblast cell line), fetal muscle, skin, foreskin and adult fallopian tube epithelial cells. Moreover, the culture of some of these lines involves feeder free/serum free systems, therefore making comparison between lines extremely difficult [Carpenter et al. (2003), Rosler et al. (2004)].

During the last 5 years, there has been an emphasis in the scientific community on improving hESC culture conditions [Carpenter et al. (2003) Carpenter et al. (2004)], undertaking hESC genetic manipulations [Imrha et al. (2004)] and optimizing differentiation protocols to produce progeny for transplantation and drug testing [Heins et al. (2004), Kehat et al. (2002)]. However, in attempting to achieve these goals, the scientific community remains seriously limited by the lack of optimized protocols to obtain relatively pure populations of specified lineages from these hESC lines using current in vitro culture conditions and procedures. This may be due to a lack of quality controls and initial variability (or lack of uniformity) in these hESC lines. Indeed, only a handful of studies have examined these parameters in order to attempt to achieve uniformity in lineage selections [Carpenter et al. (2003), Carpenter et al. (2004), Heins et al. (2004)].

The selection criteria currently used for the quality control of hESC lines are i) a typical phenotype (with high nucleo-cytoplasmic ratio), ii) surface markers (SSEA3, SSEA 4, TRA1-60, TRA1-80, GTCM2, TGT3430), iii) intracellular markers (Nanog, OCT4, REX1), iv) high telomerase activity, v) pluripotency in vitro and in vivo, and vi) an ability to sustain cryopreservation, with maintenance of these characteristics over an extended period of propagation.

It has been demonstrated that even hESC lines fully characterized against the above criteria show variability of gene expression [Abeyta et al. (2004)] and the potential of these lines to differentiate into different lineages under in vitro conditions is highly variable [Richards et al. (2002)]. It has been observed that although some hESC lines can be maintained for prolonged periods of time without losing stem cell characteristics, quantitative analysis of antigen expression by flow cytometry and gene expression by microarray suggests subtle differences in the expression of small subsets of genes upon long-term culture [Abeyta et al. (2004), Kelly and Rizzino (2000)], including a gain of chromosomes 17 q and 12 [Draper et al. (2004)].

The ability of a typical hESC colony to show clonal expansion despite the heterogeneous nature of cells present may be an important criterion to define pluripotency in these cells, even though clonal efficiency may be very low [Heins et al. (2004)]. The current conditions and procedures used for deriving these clones from hESC lines are far from optimal. To date only a few single-cell clones from the parental hESC lines, H1, H9, H13,H16 and J3 have been described. In each case these were achieved by physically picking of single cells under the microscope, with a maximum clonal efficiency of 0.83% [Amit et al. (2000)]. This procedure for clonal derivation from hESC lines is very labour-intensive and highly subjective.

Most hESC lines previously described are not clonally derived and hence pluripotency may be restricted to a small subpopulation. Consequently, the possibility exists that within apparently homogeneous populations of hESC colonies there exist multipotent precursor cells of different lineages forming multiple germ layers.

The present invention is predicated on the employment of fluorescence activated cell sorting (FACS) for the successful derivation of new human embryonic stem cell (hESC) clones. Accordingly, the present invention relates to the inventors' novel methodology for isolating individual cells and clones from stem cell lines, to cells and clones isolated by the method, and to applications thereof.

### SUMMARY OF THE INVENTION

According to a first aspect of the present invention there is provided a method for isolating individual viable stem cells from a stem cell line, wherein said method comprises:

(a) contacting cells comprising the stem cell line with at least one fluorescent marker;

(b) subjecting the cells to fluorescence activated cell sorting (FACS); and

(c) obtaining one or more individual viable stem cells sorted by the FACS.

The fluorescent marker may comprise at least one extracellular marker. The at least one extracellular marker may comprise a fluorescent tag conjugated to an antibody specific for any one or more of SSEA-1, SSEA-3, SSEA-4, TRA-1-60 or TRA-1-81. The fluorescent tag may comprise any one or more of fluorescein isothiocyanate (FITC), phycoerythrin (PE), peridinin-chlorophyll-protein complex (PerCP), tricolour (TC), texas red, allophycocyanin (APC), or Synergy Brands (SYBR) green.

The fluorescent marker may comprise at least one intracellular marker. The at least one intracellular marker may comprise a fluorescent tag selected from the group comprising green fluorescent protein (GFP), carboxyfluorescein diacetate (CFDA), carboxyfluorescein diacetate succinimidyl ester (CFSE), 7-amino-actinomycin D (7MD) or propidium iodide (PI).

Additionally or alternatively, the at least one intracellular marker may comprise a fluorescent tag conjugated to a nucleic acid probe specific for Nanog or OCT4. The fluorescent tag may comprise SYBR green.

In one embodiment, the fluorescent markers are intracellular markers, comprising carboxyfluorescein diacetate (CFDA) and propidium iodide (PI).

The FACS may comprise gating cells comprising the stem cell line on cell size and forward scatter.

The stem cell line may be an embryonic stem cell line. The embryonic stem cell line may be a human embryonic stem cell line. The human embryonic stem cell line may be the human embryonic stem cell line designated ESI-hES3 or the line designated Endeavour 1 deposited with the China Centre for Type Culture Collection (CCTCC) on 6 Jan. 2006 under Accession number C200602.

According to a second aspect of the present invention there is provided a method for deriving one or more clones from a stem cell line, wherein said method comprises:

(a) contacting cells comprising the stem cell line with at least one fluorescent marker;

(b) subjecting the cells to fluorescence activated cell sorting (FACS); and

(c) obtaining one or more individual clones sorted by the FACS.

According to a third aspect of the present invention, there is provided an individual viable cell derived from the stem cell line by the method of the first aspect.

According to a fourth aspect of the present invention, there is provided a clone derived from the stem cell line by the method of the second aspect.

In a preferred embodiment of the third or fourth aspects, the individual viable cell or clone, respectively, displays any one or more of the characteristics selected from the group comprising:

(a) differentiation potential;

(b) continuous division for long periods of time;

(c) cell surface expression of:
(i) the stage-specific embryonic antigens (SSEAs) SSEA-1, SSEA -3, SSEA-4;
   (ii) the tumor recognition antigens (TRAs) TRA-1-60 and TRA-1-81;

(d) expression of OCT4;

(e) an intracellular expression pattern characteristic of pluripotency;

(f) expression of nanog or OCT4 mRNA;

(g) embryonic body formation; or

(h) teratoma formation consisting of highly differentiated cells and tissues derived from all three germ layers, after injection of the hESC clone under kidney capsules of NOD-SCID mice.

According to a fifth aspect of the present invention there is provided a human embryonic stem cell (hESC) clone, designated hES 3.1 and deposited pursuant to the Budapest Treaty with the China Centre for Type Culture Collection (CCTCC) on 6 Jan. 2006 under Accession number ______.

According to a sixth aspect of the present invention there is provided an hESC clone designated hES 3.2 and deposited pursuant to the Budapest Treaty with the China Centre for Type Culture Collection (CCTCC) on 6 Jan. 2006 under Accession number ______.

According to a seventh aspect of the present invention there is provided an hESC clone designated hES 3.3 and deposited pursuant to the Budapest Treaty with the China Centre for Type Culture Collection (CCTCC) on 6 Jan. 2006 under Accession number ______.

According to an eighth aspect of the present invention there is provided an hESC clone designated E1C1 and deposited pursuant to the Budapest Treaty with the China Centre for Type Culture Collection (CCTCC) on ______ under Accession number ______.

According to a ninth aspect of the present invention there is provided an hESC clone designated E1C2 and deposited pursuant to the Budapest Treaty with the China Centre for Type Culture Collection (CCTCC) on ______ under Accession number ______.

According to a tenth aspect of the present invention there is provided an hESC clone designated E1C4 and deposited pursuant to the Budapest Treaty with the China Centre for Type Culture Collection (CCTCC) on ______ under Accession number ______.

According to an eleventh aspect of the present invention, there is provided a method for producing a differentiated cell from a cell of the third aspect or clone of any one of the fourth to the seventh aspects, wherein said method comprises:

(a) co-culturing the cell or clone with feeder cells;

(b) contacting the cell or clone with a differentiation factor; and

(c) culturing the cell or clone under conditions suitable to induce differentiation.

Optionally, the method may further comprise:

(d) screening the differentiated cells for characteristics of the differentiated cell; and

(e) separating substantially the differentiated cells from any undifferentiated cells.

According to a twelfth aspect of the present invention, there is provided a differentiated cell produced by the method of the eleventh aspect.

The differentiated cell may be characteristic of a vascular cell, a heart cell, a nerve cell, a lung cell, a kidney cell, a liver cell, a spleen cell, an epithelial cell or a pancreatic cell. In one embodiment the differentiated cell may be characteristic of a pancreatic cell. The differentiated cell may be an insulin-producing cell.

According to a thirteenth aspect of the present invention, there is provided a method for treating a disease in a subject, wherein said method comprises administering to the subject a cell of the third aspect, a clone of any one of the fourth to tenth aspects or a differentiated cell of the twelfth aspect. The disease may be diabetes.

According to a fourteenth aspect of the present invention, there is provided use of a cell of the third aspect, a clone of any one of the fourth to tenth aspects and/or a differentiated cell of the twelfth aspect in the manufacture of a medicament for the treatment of a disease.

According to a fifteenth aspect of the present invention, there is provided a method for proliferating a cell of the third aspect or a clone of any one of the fourth to the tenth aspects in an undifferentiated form, wherein said method comprises co-culturing the cell or clone with feeder cells. The feeder cells may be human embryonic fibroblast feeder cells.

### Definitions

In the context of this specification, the term “comprising” means “including principally, but not necessarily solely”. Furthermore, variations of the word “comprising”, such as “comprise” and “comprises”, have correspondingly varied meanings.

The term “expression” as used herein refers interchangeably to expression of a gene or gene product, including the encoded protein.

The term “fluorescent marker” as used herein refers to any marker that may be capable of fluorescing when excited by light of a particular wavelength or wavelength range. A fluorescent marker may facilitate detection of a cellular molecule, such as a protein, polypeptide or nucleic acid. For example, a fluorescent marker may comprise a fluorophore or fluorescent tag conjugated either directly or indirectly to an antibody specific for an intracellular, extracellular or cell surface-associated molecule.

As used herein the term “clone” means a group of genetically identical cells derived from a single ancestral cell.

The term “differentiation factor” as used herein refers to a molecule or compound, natural or synthetic, capable of inducing or promoting the differentiation of a pluripotent cell into a specialized form.

As used herein the terms “treating” and “treatment” refer to any and all uses which remedy a condition or symptoms, prevent the establishment of a condition or disease, or otherwise prevent, hinder, retard, ameliorate or reverse the progression of a condition or disease or other undesirable symptoms in any way whatsoever.

As used herein the term “disease” refers to any disease, disorder or ailment, including but not limited to infectious, non-infectious and/or degenerative diseases.

### BRIEF DESCRIPTION OF THE DRAWINGS

Embodiments of the present invention will now be described, by way of example only, with reference to the accompanying drawings:

FIG. 1. Gross morphology of hES colonies (right two panels, 100× and 200× magnification) and alkaline phosphatase localization (left panel, 100× magnification) in clones hES 3.1, 3.2, and 3.3 and parent line ESI-hES 3.

FIG. 2. Embryoid body formation, from top to bottom, by clone 3.1, 3.2 and 3.3 (left panel) in vitro differentiation to different cell types (middle panel) and gene expression for ectoderm (nestin), mesoderm (renin) endoderm (α-fetoprotein) and control (β-actin)(right panel).

FIG. 3. Immunolocalisation of the stem cell surface markers OCT4, SSEA 3, SSEA4, TRA-1-81 and TRA-1-60 in clone hES 3.1. A similar expression of these surface markers was observed in other clones.

FIG. 4. RT-PCR expression of undifferentiating marker, nanog, and differentiating markers, nestin (ectoderm), α-fetoprotein (endoderm) and renin (mesoderm), with corresponding molecular masses on left side as compared to the control gene β-actin.

FIG. 5. Karyotype of clones (a) hES 3.1, (b) hES 3.2 (c) hES 3.3, and of parent cell line ESI-hES 3 (d).

FIG. 6. Formation of teratoma by hES clone 3.1 in NOD SCID mice. (a) gut-like structure (endoderm); (b) cartilage-like structure (arrow, mesoderm); (c) blood vessel-like (endothelial); (d) neural rosette-like structures (arrows, ectoderm). Similar structures were also observed in teratoma formed by clones hES 3.2 and 3.3 (data not shown).

FIG. 7. Gross morphology of hES clones (E1C1P1, E1C2P1, E1C3P1, E1C4P1) from Endeavour-1 (E1). “P[X]”: passage number.

FIG. 8. Immunolocalisation (left panels) and semi-quantitative expression (right panels) of (A) BTIII, (B) AFP and (C) CD34 as markers for ectoderm, endoderm and mesoderm, respectively.

Immunolocalisation of these markers in each block from top to bottom in Endeavour-1 (E1), E1C1, E1C2, E1C3, E1C4 and from left to right, nuclear staining with DAPI, middle, specific localization of marker and right, merger.

FIG. 9. Immunolocalisation of stem cell surface markers. From left: SSEA4, TRA-1-60 and TRA-1-81 in parent line E1 and four clones, E1C1, E1C2, E1C3, E1C4. “P[X]”: passage number.

FIG. 10. RT-PCR expression of undifferentiating marker, nanog and differentiating markers, nestin (ectoderm), α-fetoprotein (AFP) (endoderm) and rennin (mesoderm) and markers on the left side and house keeping gene, β-actin.

FIG. 11. Karyotype of the parent line, Endeavour-1 (E1P19), and clonal lines, E1C1P4, E1C2P4, E1C3P4 and E1C4P4. The karyotype of E1C3P4 shows translocation. “P[X]”: passage number.

FIG. 12. Teratoma formation after injecting under the kidney capsule of SCID mice by Endeavour-1 (E1P5) and its clones (E1C1P4, E1C3P4, E1C4P4). E1C2P4 also showed a similar teratoma formation (histology not shown). “P[X]”: passage number.

### BEST MODE OF PERFORMING THE INVENTION

The present invention provides for the derivation of viable individual cells and clones from a parent stem cell line by the application of fluorescence activated cell sorting (FACS). The methods described are efficient and objective. As exemplified herein the methods have been applied to the successful isolation of three new clones designated hES 3.1, hES3.2 and hES 3.3 from the ESI-hES3 human embryonic stem cell line, and a further four new clones designated E1C1, E1C2, E1C3 and E1C4 from the human embryonic stem cell line Endeavour-1, deposited with the China Centre for Type Culture Collection (CCTCC) on 6 Jan. 2006 under Accession number C200602. Of these clones, hES 3.1, hES3.2, hES 3.3, E1C1, E1C2 and E1C4 have been deposited pursuant to the Budapest Treaty with the China Centre for Type Culture Collection (CCTCC) under the following Accession numbers.

| | | |
|---|---|---|
| Clone Designation | Date Deposited | Accession Number |

| | |
|---|---|
| hES 3.1 | 6 Jan. 2006 |
| hES 3.2 | 6 Jan. 2006 |
| hES 3.3 | 6 Jan. 2006 |
| E1C1 | |
| E1C2 | |
| E1C4 | |

The derivation of new clones by these methods along with authentic profiling of all available hESC lines for genetic, epigenetic, chromosomal, molecular and biological characteristics is very pertinent for achieving uniform lineage specifications for future transplantation therapies.

Those skilled in the art will readily appreciate that the methods described herein may be applied to isolating individual viable cells, or deriving viable clones, from any stem cell line, for example, adult or embryonic, and that the derivation of the new clones as disclosed herein is merely an indicative example of the general application of fluorescence activated cell sorting (FACS) for this purpose.

Accordingly, the present invention provides for methods of isolating individual viable cells from a stem cell line, comprising contacting cells comprising the stem cell line with at least one is fluorescent marker, subjecting the cells to fluorescence activated cell sorting (FACS) and obtaining one or more individual viable stem cells sorted by the FACS.

Similarly, the present invention also provides for methods of deriving one or more clones from a stem cell line, comprising contacting cells comprising the stem cell line with at least one fluorescent marker, subjecting the cells to fluorescence activated cell sorting (FACS) and obtaining one or more individual viable clones sorted by the FACS.

Suitable fluorescent markers may facilitate detection of one or more cellular molecules, such as proteins, polypeptides or nucleic acids. For example, a fluorescent marker may comprise a fluorophore or fluorescent tag conjugated either directly or indirectly to an antibody specific for an intracellular, extracellular or cell surface-associated molecule. Examples of fluorophores or fluorescent tags suitable for use in accordance with the present invention include, but are not limited to, fluorescein isothiocyanate (FITC), phycoerythrin (PE), peridinin-chlorophyll-protein complex (PerCP), tricolour (TC), texas red, allophycocyanin (APC), or Synergy Brands (SYBR) green. Methods of indirect conjugation may include conjugating a streptavidin-linked fluorophore or fluorescent tag to a biotin-labelled marker. Examples of cell surface markers include, but are not limited to, the stage-specific embryonic antigens (SSEAs) SSEA-1, SSEA-3, SSEA-4 or the tumor recognition antigens (TRAs) TRA-1-60 and TRA-1-81. Examples of intracellular fluorescent markers include, but are not limited to, green fluorescent protein (GFP), carboxyfluorescein diacetate (CFDA), carboxyfluorescein diacetate succinimidyl ester (CFSE), 7-amino-actinomycin D (7AAD) or propidium iodide (PI). Fluorescent markers may be used in a variety of different methods, including but not limited to, flow cytometry, fluorescence activated cell sorting (FACS), enzyme-linked immunoadsorbant assays (ELISAs), microscopy, luminometry or polymerase chain reaction (PCR).

Pluripotent ESCs are capable of both differentiating into specialized cells and dividing continuously for long periods of time. They can be defined using various established criteria, and characteristically display particular cell surface antigens including the stage-specific embryonic antigens (SSEAs) SSEA-1, SSEA -3, SSEA4, the tumor recognition antigens (TRAs) TRA-1-60 and TRA-1-81 and the POU-domain transcription factor OCT-4. The pluripotent intracellular marker, alkaline phosphatase, is also indicative of ESCs. In addition, ESCs characteristically express the pluripotent mRNA markers Nanog and OCT4, and particular differentiation markers for ectoderm (Nestin), mesoderm (Renin) and endoderm (α-fetoprotein and GATA6).

Furthermore, embryonic body (EB) formation can be observed in suspension cultures, with EBs characteristically differentiating into various cell types in vitro, thereby indicating pluripotency.

To assess in vivo pluripotency, ESCs can, for example, be injected under the kidney capsule of NOD-SCID mice.

Previously described hESC lines, although appearing phenotypically similar and considered homogeneous population of cells expressing stem cell surface markers, were not clonally derived. Therefore, pluripotency of such lines could be demonstrated only in small populations rather than in individual cells. In contrast, the inventors have herein demonstrated that individual hES cells in each of the newly described clones are pluripotent, being in continuous culture with continued demonstration of developmental potential in vivo.

The stable maintenance of diploid chromosome numbers in all exemplary clones indicates that these clones have a stable karyotype even after prolonged culture and after repeated freezing/thawing cycles. The clones also formed teratomas when injected under the kidney capsule of NOD-SCID mice, with teratomas displaying different tissues derived from all three germ layers indicating pluripotency in vivo. The clones also formed embryoid bodies that, after seeding in culture plates, formed cells of different lineages, thereby further demonstrating pluripotency in vitro.

Taken together, these results suggest that these newly established clones from the parent lines ESI-hES 3 and Endeavour-1 have similar properties as reported for other hES lines.

Accordingly, the present invention provides for individual viable cells isolated from stem cell lines by the methods described above.

Similarly, the present invention also provides for clones derived from stem cell lines by the methods described above.

Using the established criteria for characterizing pluripotency discussed above, the inventors have described herein the derivation and characterization of three human embryonic stem cell clones, designated hES 3.1, 3.2 and 3.3, from the parent line ESI-hES 3, and a further four new clones designated E1C1, E1C2, E1C3 and E1C4 from the human embryonic stem cell line Endeavour-1, by the application of FACS analysis upon single cell preparations. The efficiency of cloning was low (<0.5%) but comparable to that reported for other hESC lines which use physical transfer of hESC under a microscope, being a strategy that does not ensure single cell transfer.

Accordingly, the present invention provides for clones isolated from a human embryonic stem cell (hESC) line, wherein the hESC clone is designated hES 3.1, hES 3.2, hES 3.3, E1C1, E1C2, E1C3 or E1C4. Development of the methods described herein for propagation of hESC provide significant flexibility in handling a wide variety of hESC cultures containing heterogeneous mixtures of differentiated and undifferentiated hES colonies. These procedures appear in stark contrast to those previously applied, wherein for example the ESI-hES 3 line was previously propagated by a labour-intensive physical dissection in an organ culture plate that can house only 6-8 colonies involving concomitantly increased labour costs.

The inventors have also demonstrated that resuspending hES single cells in SR conditioned medium from HFF maintained >98% of these cells viable after FACS analysis. Similarly concentrated conditioned medium from hES cells grown in the presence of FCS has been shown to promote cell survival and maintenance of an undifferentiated fate in newly-created hESC lines.

Accordingly, the present invention provides methods for proliferating the sorted cells or clones as described above in an undifferentiated form, comprising co-culturing the cells or clones with a feeder cell.

Persons of skill in the art will further appreciate that undifferentiated embryonic stem cells may be induced to differentiate into particular cell types by the exposure of these cells to particular molecules or combinations thereof, such as retinoic acid, Wnt, Sonic Hedgehog for neural 30 differentiation and activin A for endodermal differentiation.

Accordingly, the present invention provides for methods of producing differentiated cells from the sorted cells or clones as described above, comprising for example co-culturing the cells or clones with a feeder cell, exposing the cells or clones to a differentiation factor and culturing the cells or clones under conditions suitable to induce differentiation. Optionally, the methods may further comprise screening the differentiated cells for characteristics of the differentiated cell and separating substantially the differentiated cells from any undifferentiated cells or clones.

The present invention moreover provides differentiated cells produced by the methods as described above.

By manipulating culturing conditions, cells of the present invention can be induced to differentiate into any given endodermal, mesodermal or ectodermal cell type. Techniques and methodologies for such manipulation will be known to those skilled in the art. This pluripotent capacity of cells of the invention may be utilised, for example, for the generation of cells producing a desired biomolecule. Further, differentiated cells, tissues or organs, the products of cells of the present invention, may also be used, for example, for therapeutic or prophylactic transplantation purposes, or for a range of scientific purposes such as the identification of gene targets for pharmacological agents, for generating transgenic or chimeric organisms to serve as, for example, models of specific human genetic diseases, for studying differentiation, development or other biological processes.

The range of applications of cells of the present application is in no way to be limited by the above discussion. Those skilled in the art will readily appreciate the diversity of applications of clonal cells of the invention.

By way of example only, skilled artisans will appreciate that differentiated cells produced from individual cells or clones of the invention, or cells or clones produced by methods of the invention, may be used for the treatment of a wide variety of diseases. Such treatment may comprise administering to a patient in need differentiated cells as described above. For example, human embryonic stem cell clones of the present invention may be induced to differentiate into insulin-producing cells which in turn may find application in the treatment of diabetes, such as Type I diabetes. Other potential applications are discussed, for example, in Keller (2005).

The present invention will now be further described in greater detail by reference to the following specific examples, which should not be construed as in any way limiting the scope of the invention.

### EXAMPLES

### Example 1

### Methods and Materials for Human Embryonic Stem Cell Culture

All reagents including culture media and sera were obtained from Gibco/lnvitrogen (Carlsbad, Calif. USA, www.invitrogen.com).

The human ESC line, ESI-hES3, was obtained from Embryonic Stem Cell International Pte Ltd. Singapore. The human ESC line, hES3, which constitutively expresses GFP (Envy line), was obtained from the Monash Immunology and Stem Cell Laboratories, Melbourne (courtesy Dr Andrew Elefanty). The Endeavour-1 cell line was derived by the inventors and has been deposited with the China Centre for Type Culture Collection (CCTCC) on 6 Jan. 2006 under Accession number C200602. hESC colonies were maintained in gelatin-coated six well culture plates (Becton Dickinson, N.J., USA; www.bdbiosciences.com) on gamma-irradiated (45 Gy) primary human fetal fibroblast (HFF; passage 6) feeder layers (1.5×10⁶ cells/ml) and cultured at 37° C., 5% CO₂ in serum replacer (SR) medium consisting of Dulbecco's knockout (KO-DMEM) high glucose, supplemented with 20% knockout SR (Gibco, Carlsbad, Calif. USA), 2 mM L-glutamine, 0.1 mM nonessential amino acids, 0.01 mM 2-mercaptoethanol, 1×insulin-transferrin-selenium, basic fibroblast growth factor (bFGF), 4 ng/ml, 25 U/ml penicillin and 25 μg/ml streptomycin.

This study was undertaken on hESC and HFF lines with institutional ethics approval (HREC 01270 and HREC 02247, respectively).

Routinely >75 hESC colonies were grown per well of a six well culture plate. The sub-culturing of hESC colonies, with a 1:6 split, was performed every 6-7 days using 0.05% trypsin for 2 min. Cryopreservation of clones was carried out by vitrification in open pulled straws as well as by slow freezing in cryovials according to procedures previously described (Reubinoff et al., 2001).

### Example 2

### Preparation of Single hES Cell Preparations

Aliquots of 300-400 hESC colonies, including aliquots from the Envy line, were dissected from six well plates by gently washing twice and with collagenase type IV (1 mg/ml in phosphate buffered saline (PBS) without Ca²⁺; 1 ml/well) treatment for 7 min at 37° C. hESC colonies were allowed to settle at the bottom of a 15 ml tube for 5 min and supernatant was aspirated. hESC colonies were dissociated into single cells by using 0.05% trypsin/0.25% EDTA at 37° C. for 7 min, triturated carefully twice with a pipette. Finally, cell preparations were re-suspended at 1×10⁶ cells/ml in conditioned medium collected from HFF cultured in SR medium for 24 h.

### Example 3

### FACS Sorting of hES Single Cell Preparations

A FACScalibur (Becton Dickinson, Sydney) was used to select only hESC derived from the hESC line designated ESI-hES3 by gating on size and forward-scatter (FSC). The exclusive selection of stem cells by this procedure was confirmed by using FACS sorting of a single cell preparation (see Example 2) from an Envy hES line that consitutively expresses GFP. Each cell was dispensed into a well of a 96 well plate containing HFF as a feeder layer in SR medium with 5% CO₂ at 37° C. for two weeks. The dispersion of single stem cells into each well of the 96 well plate was therefore confirmed by using a single cell preparation from the Envy hES3 line and visualization under a fluorescent microscope. The viability of single hESCs after FACS was >98% as assessed by fluorescent staining with carboxyfluorescein diacetate (CFDA) and propidium iodide (PI). Briefly, hESC were washed with 500 μL PBS at 800 rpm for 3 min and re-suspended in 250 μL CFDA (0.1 mM in DMSO) then incubated for 30 min at 37° C., washed in PBS and re-suspended in 200 μL PBS, with 10 μL PI (100 μg/ml PBS) then added and incubated on ice for 5 min before counting viable (green fluorescent) and non-viable (red fluorescent) cells under a fluorescence microscope.

Clones obtained from ESI-hES3 were initially passaged by physical dissection into 24 well plates and subsequently into six well plates by trypsin.

### Example 4

### Procedures for Characterization of Clones

### Surface and Intracellular Markers

Immunohistochemical localization of various stem cell surface markers, i.e. stage-specific embryonic antigens SSEA-1, SSEA-3, SSEA-4; tumor recognition antigens TRA-1-60, TRA-1-81, and a POU-domain transcription factor OCT-4, were carried out on clones isolated in Example 3 using primary antibodies (1:250) against these surface markers and visualized using fluorescein isothiocyanate (FITC)-conjugated appropriate secondary antibodies as per the supplier's instructions (Chemicon, VIC, Australia; www.chemicon.com.au).

The expression of these stem cell surface markers on single cell preparations was also estimated by flow cytometric analysis according to the procedure described by Carpenter et al. (2003). The pluripotent intracellular marker, alkaline phosphatase, was assessed immunohistochemically using a commercially available kit (Sigma-Aldrich) following the manufacturer's instructions.

### RT-PCR

Total RNA from hESC was extracted using an RNeasy mini kit (Qiagen) with DNase treatment. First strand cDNA was synthesized using 5 μg total RNA with MMLV-RT (Gibco) and oligo (dT) primer (Roche). Expression of the pluripotent markers Nanog and OCT4, and the differentiation markers for ectoderm (nestin), mesoderm (renin) and endoderm (α-fetoprotein+GATA6) was assessed by semi-quantitative PCR using Gel Doc System (BIO RAD). Expression levels of the markers were normalized to the control gene β-actin.

### Karyotyping

A standard G banding and multicolor spectra karyotyping (SKY) approach was undertaken with a SKY H-10 kit as per the manufacturer's instructions (Applied Spectra Imaging, Inc, Carlsbad, Calif.). For each sample, 20 metaphases were captured for modal determination.

### Transplantation into NOD-SCID Mice for Teratoma Formation

To assess in vivo pluripotency, approximately 2×10⁶ cells from each clone were injected under the kidney capsule of NOD-SCID mice. The animals were euthanased 6-8 weeks later and grafts examined histologically.

### Formation of Embryoid Bodies (EB)

hESC colonies from each clone were dissected from wells with collagenase and cultured in non-tissue culture plates (suspension culture) in SR medium for one week to produce embryoid bodies (EBs). The EBs were then seeded in tissue culture dishes and SR medium without bFGF for two weeks to induce differentiation. The expression of lineage markers in hESC cultures after RNA extraction for ectoderm, mesoderm and endoderm were evaluated by RT-PCR as described above.

### Freezing and Thawing

Clones were cryopreserved by slow and fast freezing procedures (vitrification) and thawed several times as described previously (Reubinoff et al., 2001).

### Example 5

### Derivation of hESC Clones

The procedures described above in Examples 1 to 3 were scaled up and optimized for growing large numbers of hESC colonies in 96 well plates, resulting in relatively pure single cell preparations. This approach facilitated optimization of FACS of hESC for clonal propagation. The exclusive selection of stem cells by the gating procedure described above in Example 3 was confirmed using a single cell preparation from an Envy hES line that constitutively expresses GFP, with single green fluorescent stem cells visualized under the fluorescent microscope. Three clones were obtained, designated hES 3.1, hES 3.2 and hES 3.3 after FACS of single cell preparations from the ESI-hES 3 line in 96 well plates.

Routinely it was determined that sub-culturing (splitting at a 1:6 ratio) of hESC colonies by trypsin was an optimal procedure. Seeding densities of >75 hESC colonies per well in six well plates could be propagated without significant induction of differentiation. However, for obtaining single cell preparations, hESC colonies were first treated with collagenase followed by trypsin digestion to single cell preparation. This procedure also helped to selectively eliminate differentiated hESC colonies, if any, by first scraping and washing off the differentiated colonies before lifting up the undifferentiated colonies by collagenase. The use of collagenase also helped eliminate most of the fibroblasts during sedimentation of hESC colonies. A relatively pure population of hESC with viability >98% was dispensed as a single cell per well in 96 well plates by gating on size and FSC.

In addition, the procedures described above in Examples 1 to 3 were used to derive four new clones after FACS of single cell preparations from the Endeavour-1 line in 96 well plates, with an overall efficiency of 0.5-2%. These four new clones were designated E1C1, E1C2, E1C3 and E1C4.

### Example 6

### Characterization of hESC Clones

To evaluate whether the hESC clones were stem cells, they were characterized according to their morphology, expression of pluripotent genes, stem cell surface markers and ability to differentiate both in vitro and in vivo.

### Example 6A

### Morphology, Cryopreservation and EB Formation

### 6A1. Clones Derived from ESI-hES3

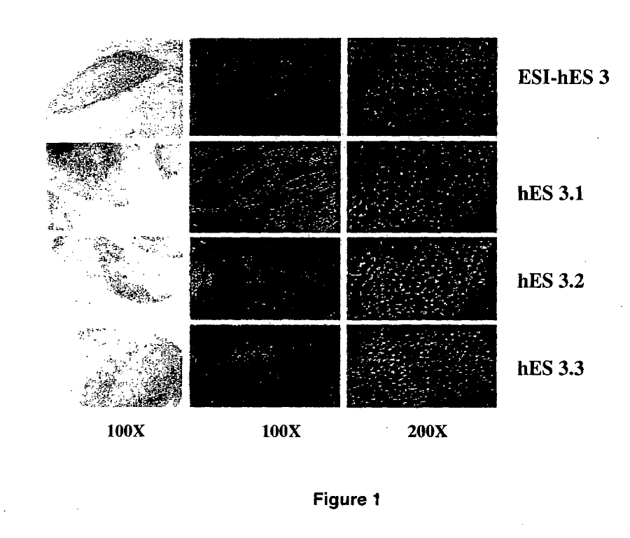

Under the culture conditions described for hESC in Example 1, all three derived clones, hES 3.1, hES 3.2 and hES 3.3 (at passage 10), and the parent line, ESI-hES 3 (at passage 155) form large compact colonies with a distinct stem cell morphology (FIG. 1).

The gross degree of spontaneous differentiation as evidenced by appearance of cobblestone morphology in colonies from each clone was found to be 5-10%, which is comparable to the parent line. However, clone hES 3.2 showed a higher degree of differentiation (>20%) if propagated after day 5.

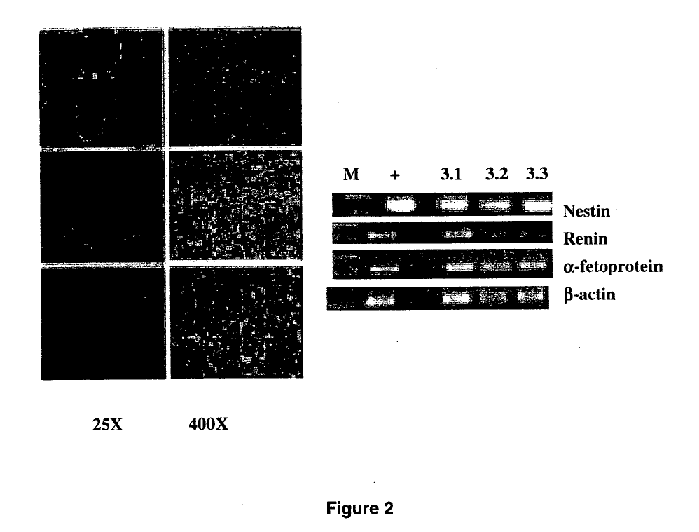

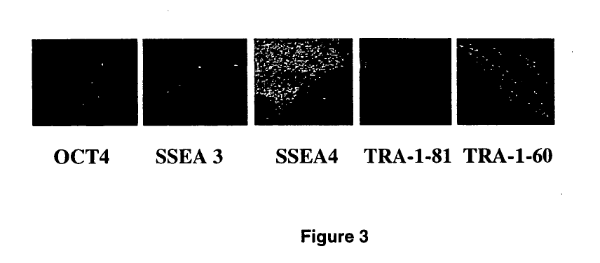

These clones were successfully cryopreserved by vitrification and slow freezing methods several times, being re-cultured with a plating efficiency of >90% and >25% respectively (data not shown). These clones also formed embryoid bodies in suspension cultures that, after seeding in culture plates, formed differentiated cell types. These cells showed marker expression for three germ layers and the disappearance of the pluripotent marker nanog during culture (FIG. 2).

### 6A2. Clones Derived from Endeavour-1

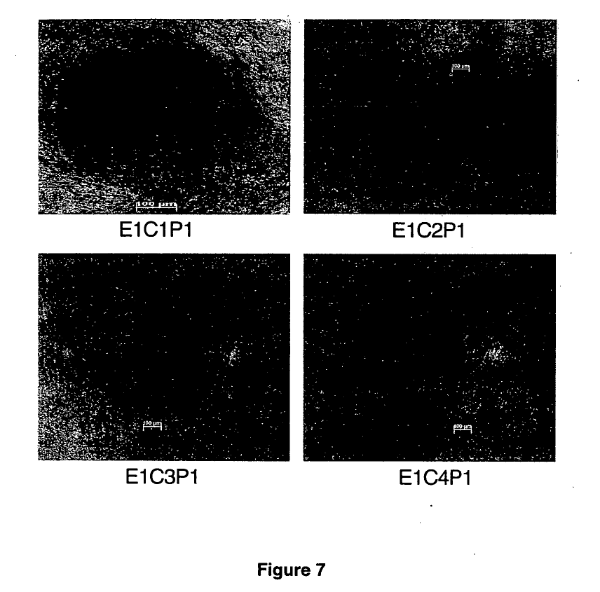

Under the culture conditions described for clones derived from ESI-hES3, the clones E1C1, E1C2, E1C3 and E1C4 form large compact colonies with a distinct stem cell morphology (FIG. 7).

These clones were successfully cryopreserved by both vitrification and slow freezing methods several times and thawed out with platting efficiency of >90% and >75% respectively (data not shown).

### Example 6B

### RT-PCR Analysis of Gene Expression

### 6B1. Clones derived from ESI-hES3

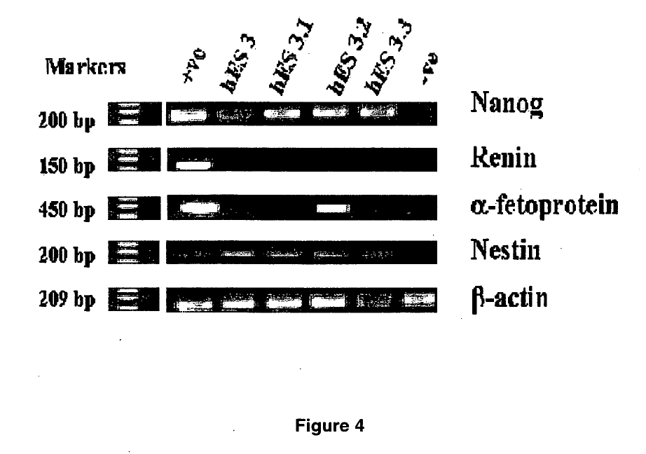

Semi-quantitative RT-PCR analysis of cDNA was carried out for expression of the pluripotent marker gene, nanog, and also the lineage marker genes, nestin (for ectoderm), renin (for mesoderm) and α-fetoprotein (for endoderm) on different batches of clones and compared with the parent line (FIG. 4).

Nanog, a marker for pluripotency, was expressed strongly in all hES clones 3.1, 3.2, 3.3 and in the ESI-hES 3 parent line. This remained so throughout the culture period for more than 4 months during successive passages. α-fetoprotein, a marker for primitive endoderm, was present in clone 3.2 but not in the other two clones or the parent cell line. Clone 3.2 also expressed the endoderm marker GATA6 (data not shown). Renin, a marker for primitive mesoderm, was not expressed in any of the clones or the parent line. Nestin, a marker for ectoderm, was seen in all hESC clones and the parent line.

### 6B2. Clones Derived from Endeavour-1

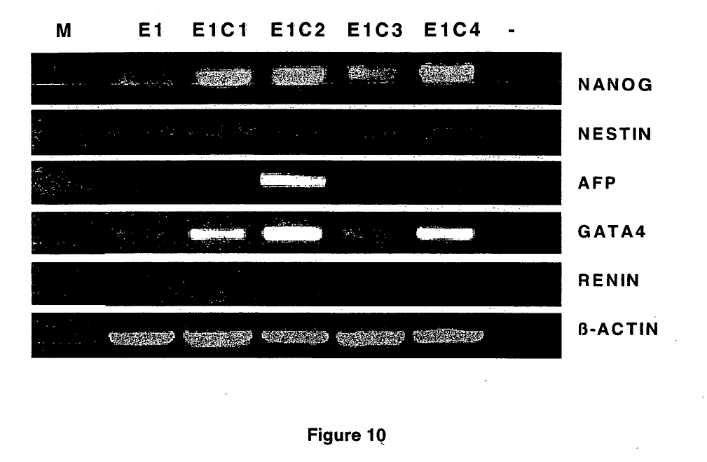

FIG. 10 shows a semi-quantitative RT-PCR analysis of cDNA carried out for expression of the pluripotent marker gene, nanog and the lineage marker genes, nestin, rennin, α-fetoprotein and GATA4 from different batches of clones as indicated and from the parent line for comparison.

As shown in FIG. 10, nanog was expressed strongly in all clones and the parent line. An endoderm marker, α-fetoprotein was present in E1C2 while another early endoderm marker, GATA4 was expressed differentially in different clones with maximum expression in E1C2. Renin, a marker for mesoderm, was not observed in any of the clones, whereas nestin, an ectoderm marker was expressed in all the clones.

### Example 6C

### Stem Cell Markers

### 6C1. Clones Derived from ESI-hES3

Table 1 summarizes the results of characterization of the three clones, hES3.1, 3.2 and 3.3. All clones, as well as the parent hESC line, ESI-hES3, showed strong expression of the surface markers OCT4, SSEA-3, SSEA-4, TRA-1-60, TRA-1-81 (FIG. 2 of hES clone 3.1 is representative of other clones), and the intracellular marker, alkaline phosphatase (FIG. 1). There was weaker expression of the surface markers SSEA-3, and OCT-4 and no expression of surface marker SSEA-1. A quantitative preliminary analysis of the stem cell surface marker TRA-1-160 by FACS indicated that the bright green fluorescing stem cells hES 3.1, 3.2 and 3.3 accounted for 92.3%, 72.5%, 59.4% respectively of the population, as compared to 65.7% in the parent line ESI-hES 3.

TABLE 1
Summary of the characteristic features of hESC clones derived from ESI-hES3

| | Markers | | | | | | |
|---|---|---|---|---|---|---|---|
| hESC/Clones | SSEA-1 | SSEA-3 | SSEA-4 | TRA-1-60 | TRA-1-81 | ALP | OCT-4 |
| ESI-hES 3 (Parent) | − | + | ++ | ++ | ++ | ++ | + |
| hES 3.1 (Clone) | − | + | ++ | ++ | ++ | ++ | + |
| hES 3.2 (Clone) | − | + | ++ | ++ | ++ | ++ | + |
| hES 3.3 (Clone) | − | + | ++ | ++ | ++ | ++ | + |

| | Gene Expression/Karyotype/Teratoma | | | | | |
|---|---|---|---|---|---|---|
| | Nanog | Nestin | Renin | α-Fetoprotein | karyotype | Teratoma |
| ESI-hES 3 (Parent) | ++ | + | − | − | 46XX | 3 Germ layers |
| hES 3.1 | ++ | + | − | − | 46XX | 3 Germ layers |
| hES 3.2 | ++ | + | − | + | 46XX | 3 Germ layers |
| hES 3.3 | ++ | + | − | − | 46XX | 3 Germ layers |

++, strong;
+, weak;
−, absent;
Nanog, Nestin, Renin, α-Fetoprotein gene expression by RT-PCR.

### 6C2. Clones Derived from Endeavour-1

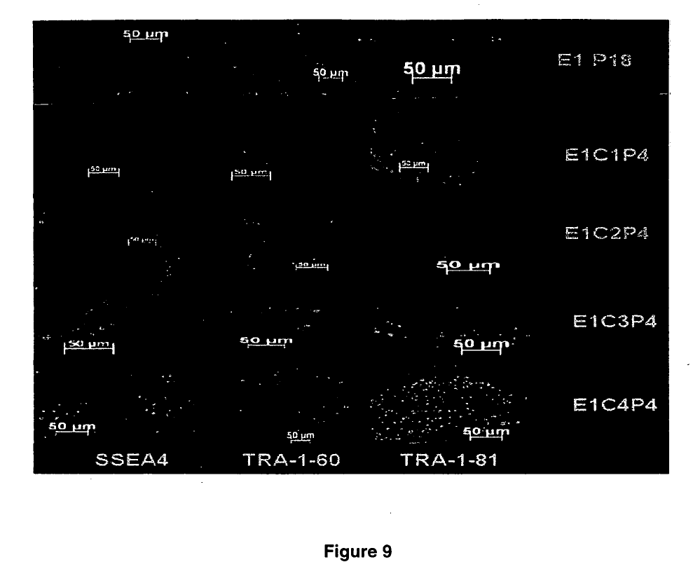

An immunolocalisation study carried out according to that described in Example 4 using various stem cell surface markers, namely, SSEA4, TRA-1-6-, TRA-1-81, demonstrated expression of these markers in Endeavour-1 and in all its clones (FIG. 9). The results of this study are summarized in Table 2.

TABLE 2
Summary of the characteristic features of hESC clones derived from Endeavour-1

| | Markers | | | | | | |
|---|---|---|---|---|---|---|---|
| hESC/Clones | SSEA-1 | SSEA-3 | SSEA-4 | TRA-1-60 | TRA-1-81 | ALP | OCT-4 |
| Endeavour-1 (Parent) | − | + | ++ | ++ | +++ | ++ | + |
| E1C1 | − | + | ++ | ++ | +++ | ++ | + |
| E1C2 | − | + | ++ | ++ | +++ | ++ | + |
| E1C3 | − | + | ++ | ++ | +++ | +++ | + |
| E1C4 | − | + | ++ | +++ | +++ | ++ | + |

| | Gene Expression/Karyotype/Teratoma | | | | | |
|---|---|---|---|---|---|---|
| | Nanog | Nestin | Renin | α-Fetoprotein | karyotype | Teratoma |
| Endeavour-1 (Parent) | + | ++ | − | − | 46XX | 3 Germ layers |
| E1C1 | + | ++ | − | − | 46XX | 3 Germ layers |
| E1C2 | + | ++ | − | ++ | 46XX | 3 Germ layers |
| E1C3 | + | ++ | − | − | 46XX | 3 Germ layers |
| E1C4 | + | ++ | − | − | 46XX | 3 Germ layers |

++, strong;
+, weak;
−, absent;
Nanog, Nestin, Renin, α-Fetoprotein gene expression by RT-PCR.

### Example 6D

### Analysis of the Ability to Differentiate

### 6D1. Pluripotency

### 6D1 (i) Clones Derived from ESI-hES3

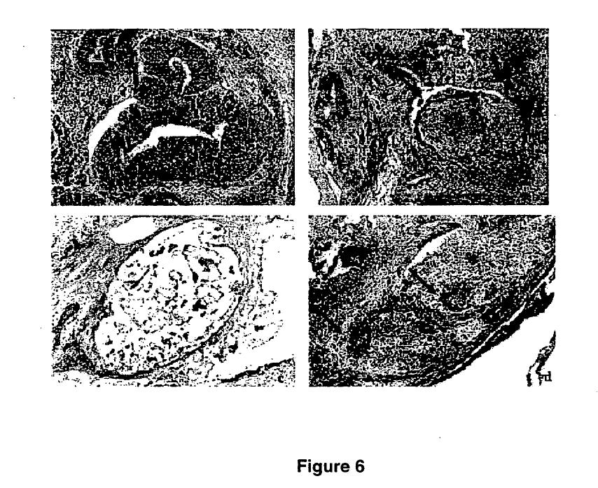

In vitro: hESCs derived from clones and the parent line also formed three-dimension embryoid bodies (EBs) in suspension cultures in vitro that expressed genes as assessed by RT-PCR encoding nestin, renin, α-fetoprotein, and markers for ectoderm, mesoderm and endoderm. The EBs could be differentiated after seeding to various cell types such as neurons (FIG. 2). In vivo: Clumps of hESC containing approximately 2×10⁶ cells each from all three clones hES 3.1, 3.2 and 3.3 at passage 10, and the parent line hES 3 at passage 150, when injected under the kidney capsule of SCID mice, formed teratomas after 4-6 weeks. The cysts containing teratomas derived from these cells consisted of highly differentiated cells and tissues derived from all three germ layers, such as gut epithelium (endoderm), cartilage-like (mesoderm) and neural rosettes (ectoderm). See FIG. 6 from clone 3.1 as a representative of each clone.

### 6D1 (ii) Clones Derived from Endeavour-1

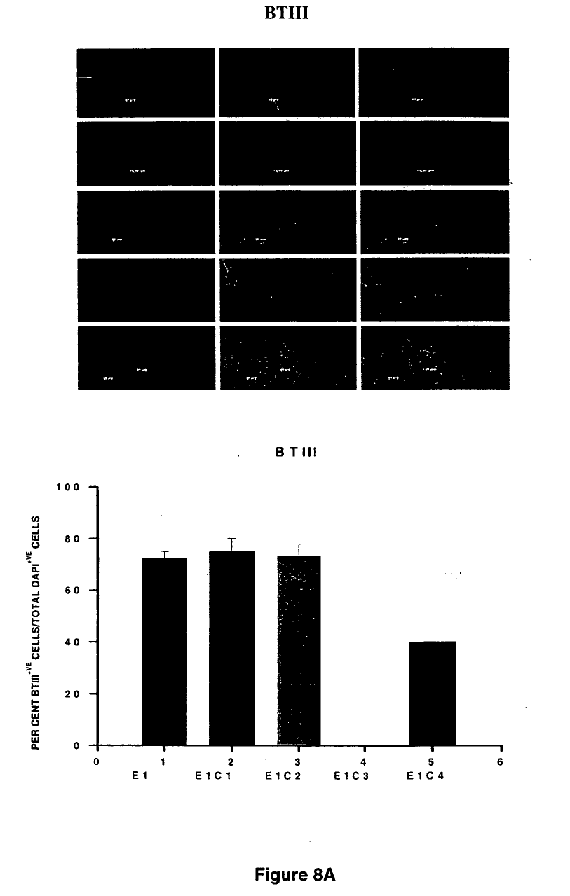

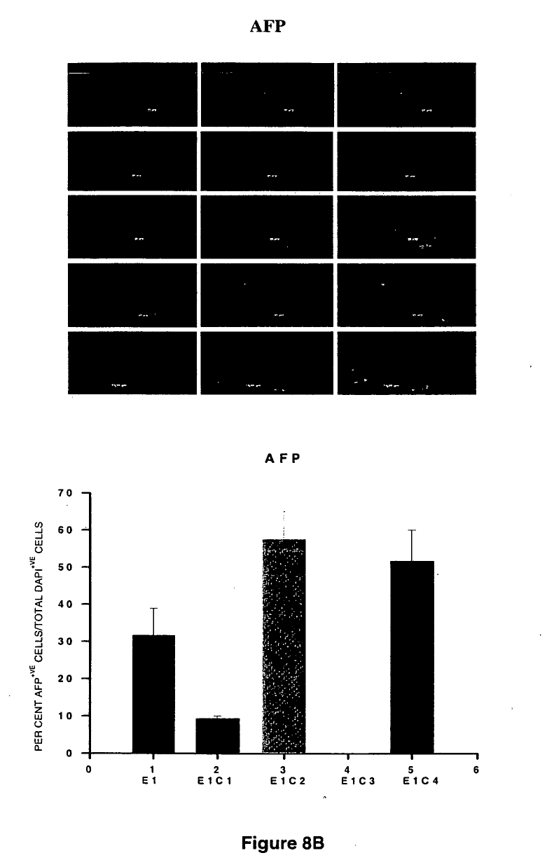

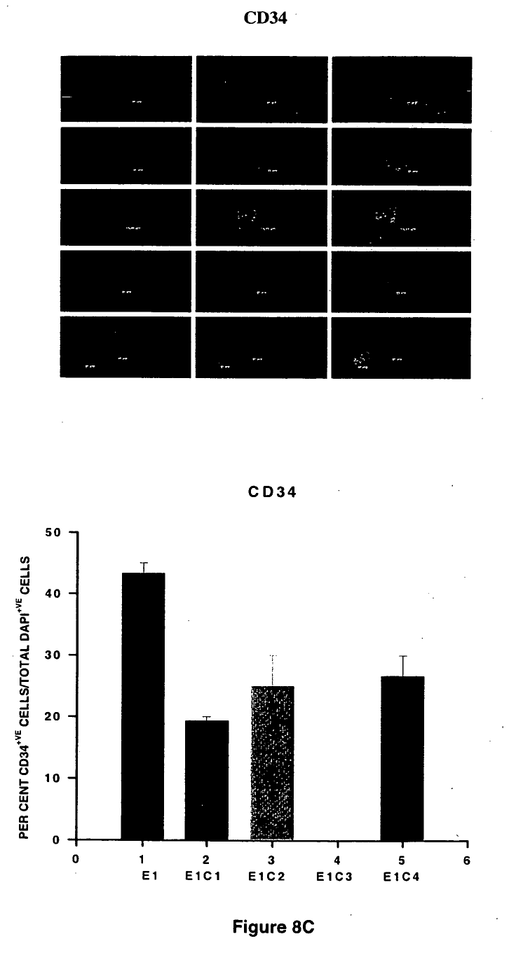

In vitro: Endeavour-1 and its clones were induced through embryoid body formation in suspension culture and upon seeding produced various cell types, all of which were derived from three germ layers, namely ectoderm, mesoderm and endoderm. A semi-quantitative analysis of the expression for specific lineage markers such as CD34, AFP and β-III tubulin showed a significant variabilities amongst different clones (FIG. 8).

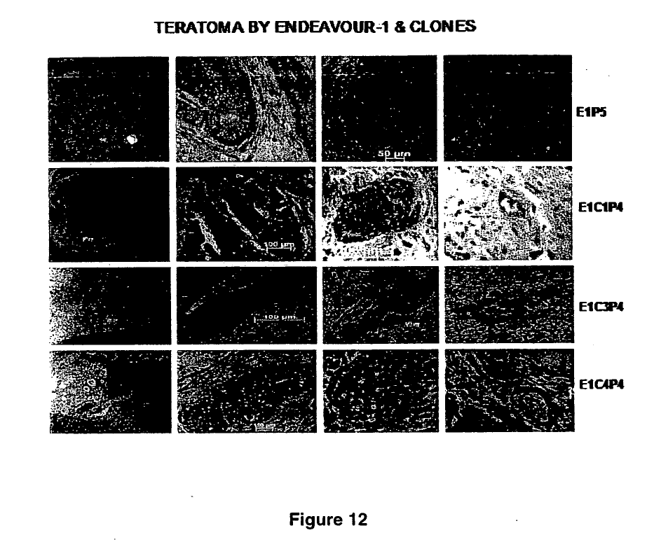

In vivo: Clumps of clones at passage 4 and of the parent line at passage 5, when injected under the kidney capsule of SCID mice, formed teratoma after 4-6 weeks. The cysts, containing teratoma derived from these cells, consisted of highly differentiated cells and tissues derived from all three germ layers, including gut epithelium (endoderm), cartilage-like material (mesoderm) and neural rosettes (ectoderm) (FIG. 12).

### 6D2. Karyotyping

### 6D2 (i) Clones Derived from ESI-hES 3

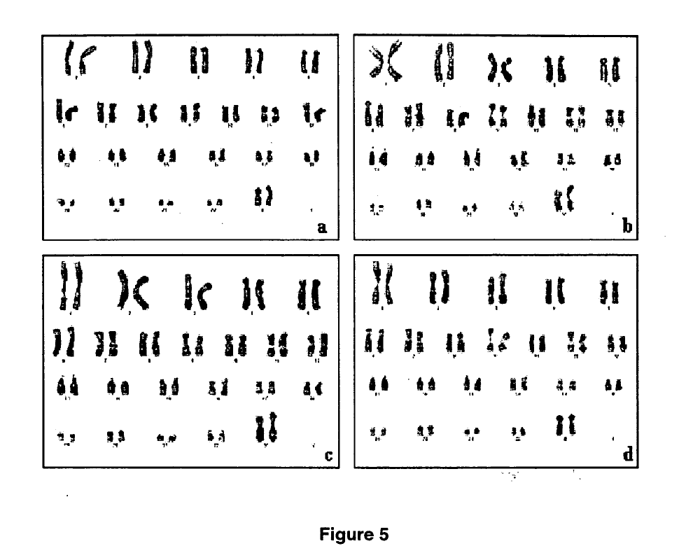

Cytogenetic evaluation of clones at passage 10 and the parent line at passage 150 by standard G-banding (20 cells for each culture) showed a normal 46 XX karyotype (FIG. 5).

### 6D2 (ii) Clones Derived from Endeavour-1

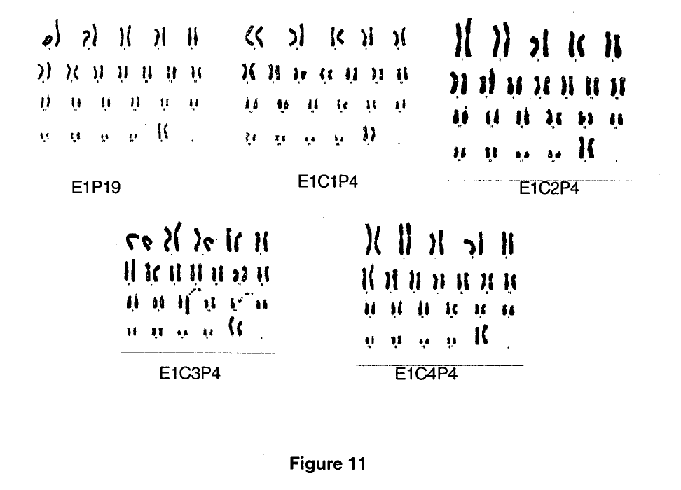

Cytogenetic evaluation of clones at passage 4 and of the parent line at passage 19 by standard G-banding (20 cells for each culture) showed a normal 46 XX karyotype, except for E1C3 which showed some reciprocal translocation involving chromosomes 15 and 17 (FIG. 11).

### REFERENCES

Abeyta M J, Clark A M, Rodriguez R T, Bodnar M S, Pera R A R and Firpo M T (2004) Unique gene expression signatures of independently derived human embryonic stem cell lines. Human Molecular Genetics 13: 601-608.
Amit M, Carpenter M K, Inukuma M S, Chiu C P, Harris C P, Waknitz M A, Itskovitz-Eldore J and Thomson J A (2000). Clonally derived human embryonic stem cell lines maintain pluripotency and proliferative potential for prolonged period of culture. Dev Biol 227: 271-278.
Carpenter M K, Rosier E, and Rao M S (2003) Characterization and differentiation of human embryonic stem cells. Cloning Stem Cells 5: 79-88.
Carpenter M K, Rosier E S, Fisk G J Brandenberger R., Ares X, Miura T, Lucero M and Rao M S (2004) Properties of four human embryonic stem cell lines maintained in a feeder-free culture system. Dev Dynamics 229: 243-253.
Draper J S, Smith K, Gokhale P, Moore H D, Meltby E, Johnson J, Meisner L, Zwaka T P, Thomson J A and Andrew P W (2004) Recurrent gain of chromosomes 17q and 12 in cultured human embryonic stem cells. Nature Biotechnology 22: 53-54.
Heins N, Englund M C O, Sjoblom C, Dahi U, Tonning A., Bergh C, Lindahl A, Hanson C, and Semb H (2004) Derivation, characterization, and differentiation of human embryonic stem cells. Stem Cells 22: 367-376.
Imreh M P, Wolbank S, Unger C, Gertow K, Aints A, Szekes A, Imreh S, Hovatta O, Fried G, Dilber S and Ahrlund-Richter L (2004) Culture and expression of human embryonic stem cell line HS 181, evaluated in a double-colour system Stem Cells Dev 13:337-343.
Kehat I, Gepstein A, Spira A, Itskovitz-Eldore J and Gepstein L (2002) High-resolution electrophysiological assessment of human embryonic stem cell-derived cardiomyocytes: a novel in vitro model for the study of conduction Circulation Res 91:659-661.
Keller G (2005) Embryonic stem cell differentiation: emergence of a new era in biology and medicine Genes Dev 19:1129-1155.
Kelly D L, Rizzino A (2000). DNA microarray analysis of genes regulated during the differentiation of embryonic stem cells. Mol Reprod Dev 56: 113-123.
Reubinoff B E, Pera M F, Vajta G and Trounson A (2001). Effective cryopreservation of human stem cells by the open pulled straw vitrification method. Human Reproduction 16: 2187-2194.
Richards M, Fong C Y, Chan W K, Wong P C and Bongso A (2002). Human feeders support prolonged undifferentiated growth of human inner cell masses and embryonic stem cell lines. Nature Biotech 20: 933-936.
Rosler E S, Fisk G J, Ares X, Irving J, Miura T, Rao M S, and Carpenter M K (2004) Long-term culture of human embryonic stem cells in feeder-free conditions. Dev Dynamics 229: 259-274.

1. A method for isolating individual viable stem cells from a stem cell line, wherein said method comprises:
(a) contacting cells comprising the stem cell line with at least one fluorescent marker;
(b) subjecting the cells to fluorescence activated cell sorting (FACS); and
(c) obtaining one or more individual viable stem cells sorted by the FACS.

2. The method according to claim 1, wherein the fluorescent marker comprises at least one extracellular marker and/or at least one intracellular marker.

3. The method according to claim 2, wherein the extracellular marker comprises a fluorescent tag conjugated to an antibody specific for any one or more of SSEA-1, SSEA-3, SSEA-4, TRA-1-60 or TRA-1-81.

4. The method according to claim 3, wherein the fluorescent tag comprises any one or more of fluorescein isothiocyanate (FITC), phycoerythrin (PE), peridinin-chlorophyll-protein complex (PerCP), tricolour (TC), texas red, allophycocyanin (APC), or Synergy Brands (SYBR) green.

5. The method according to claim 1, wherein said one or more individual viable stem cells sorted by the FACS comprises at least one clone, and wherein said at least one clone is capable of being individually cultured to form a clonal stem cell line.

6. The method according to claim 2, wherein the intracellular marker comprises a fluorescent tag selected from the group comprising green fluorescent protein (GFP), carboxyfluorescein diacetate (CFDA), carboxyfluorescein diacetate succinimidyl ester (CFSE), 7-amino-actinomycin D (7AAD) or propidium iodide (PI).

7. The method according to claim 2, wherein the intracellular marker comprises a fluorescent tag conjugated to a nucleic acid probe specific for Nanog or OCT4.

8. (canceled)

9. The method according to claim 2, wherein the FACS comprises gating cells comprising the stem cell line on cell size and forward scatter.

10. The method according to claim 1, wherein the stem cell line comprises an embryonic stem cell line.

11. The method according to claim 10, wherein the embryonic stem cell line is a human embryonic stem cell line.

12. The method according to claim 11, wherein the human embryonic stem cell line is the human embryonic stem cell line designated ESI-hES3.

13. The method according to claim 11, wherein the human embryonic stem cell line is the line designated Endeavour 1 deposited with the China Centre for Type Culture Collection (CCTCC) on 6 Jan. 2006 under Accession number C200602.

14. The method of claim 5, wherein said method comprises co-culturing one clone with feeder cells to produce a clonal stem cell line.

15. An individual viable cell derived from the stem cell line by the method according to claim 1.

16. The individual viable cell according to claim 15, wherein the cell displays any one or more of the characteristics selected from the group comprising:
(a) differentiation potential;
(b) continuous division for long periods of time;
(c) cell surface expression of:
(i) the stage-specific embryonic antigens (SSEAs) SSEA-1, SSEA -3, SSEA-4;
(ii) the tumor recognition antigens (TRAs) TRA-1-60 and TRA-1-81;
(d) expression of OCT4;
(e) an intracellular expression pattern characteristic of pluripotency;
(f) expression of nanog or OCT4 mRNA;
(g) embryonic body formation; and
(h) teratoma formation consisting of highly differentiated cells and tissues derived from all three germ layers, after injection of the hESC clone under kidney capsules of NOD-SCID mice.

17. A clone derived from the stem cell line by the method according to claim 5.

18. The clone according to claim 17, wherein the clone displays any one or more of the characteristics selected from the group comprising:
(a) differentiation potential;
(b) continuous division for long periods of time;
(c) cell surface expression of:
(i) the stage-specific embryonic antigens (SSEAs) SSEA-1, SSEA -3, SSEA-4;
(ii) the tumor recognition antigens (TRAs) TRA-1-60 and TRA-1-81;
(d) expression of OCT4;
(e) an intracellular expression pattern characteristic of pluripotency;
(f) expression of nanog or OCT4 mRNA;
(g) embryonic body formation; and
(h) teratoma formation consisting of highly differentiated cells and tissues derived from all three germ layers, after injection of the hESC clone under kidney capsules of NOD-SCID mice.

19. A human embryonic stem cell (hESC) clone, selected from the group consisting of:
the clone designated hES 3.1 and deposited pursuant to the Budapest Treaty with the China Centre for Type Culture Collection (CCTCC) under Accession number C200601:
the clone designated hES 3.2 and deposited pursuant to the Budapest Treaty with the China Centre for Type Culture Collection (CCTCC) under Accession number C200624;
the clone designated hES 3.3 and deposited pursuant to the Budapest Treaty with the China Centre for Type Culture Collection (CCTCC) under Accession number C200625:
the clone designated E1C1 and deposited pursuant to the Budapest Treaty with the China Centre for Type Culture Collection (CCTCC) under Accession number C200626;
the clone designated E1C2 and deposited pursuant to the Budapest Treaty with the China Centre for Type Culture Collection (CCTCC) under Accession number C200627; and
the clone designated E1C4 and deposited pursuant to the Budapest Treaty with the China Centre for Type Culture Collection (CCTCC) under Accession number C200628.

20.-23. (canceled)

24. A method for producing a differentiated cell, wherein said method comprises:
(a) co-culturing a viable cell of claim 15 with feeder cells;
(b) contacting the viable cell or its progeny with a differentiation factor; and
(c) culturing the viable cell or its progeny under conditions suitable to induce differentiation.

25. A method for producing a differentiated cell, wherein said method comprises:
(a) co-culturing a clone of claim 17 with feeder cells;
(b) contacting the clone with a differentiation factor; and
(c) culturing the clone under conditions suitable to induce differentiation.

26. The method according to claim 24, further comprising:
(d) screening the differentiated cells for characteristics of the differentiated cell; and
(e) separating substantially the differentiated cells from any undifferentiated cells.

27. The method according to claim 26, wherein said method further comprises genetic manipulation of the cell or clone of (a) or of the differentiated cells.

28. A differentiated cell produced by the method according to claim 24.

29. The differentiated cell according to claim 28, wherein said cell is characteristic of a vascular cell, a heart cell, a nerve cell, a lung cell, a kidney cell, a liver cell, a spleen cell, an epithelial cell or a pancreatic cell.

30. The differentiated cell according to claim 29, wherein the differentiated cell is characteristic of a pancreatic cell and is an insulin-producing cell.

31. A method for treating a disease in a subject, wherein said method comprises administering to the subject the cell according to claim 30.

32. The method according to claim 31, wherein the disease is diabetes.

33. (canceled)

34. A method for proliferating the cell according to claim 15, in an undifferentiated form, wherein said method comprises co-culturing the cell or clone with feeder cells.

35. The method according to claim 34, wherein the feeder cells are human embryonic fibroblast feeder cells.
